(19) United States
(12) Patent Application Publication
(10) Pub. No.: US 2022/0324827 A1
(43) Pub. Date: Oct. 13, 2022

(54) EXTRACTION METHOD OF PROANTHOCYANIDIN FROM CHINESE BAYBERRY (MYRICA RUBRA SIEB. ET ZUCC) LEAVES

(71) Applicant: ZHEJIANG UNIVERSITY, Hangzhou (CN)

(72) Inventors: Shiguo CHEN, Hangzhou (CN); Haibo PAN, Hangzhou (CN); Wenyang TAO, Hangzhou (CN); Xingqian YE, Hangzhou (CN)

(21) Appl. No.: 17/765,359

(22) PCT Filed: Aug. 25, 2020
(86) PCT No.: PCT/CN2020/111145

§ 371 Date: Mar. 30, 2022

(30) Foreign Application Priority Data
Jun. 3, 2020 (CN) 202010494908.8

(51) Int. Cl. C07D 311/62; B01D 11/02; B01D 21/26

(52) U.S. Cl. CPC C07D 311/62; B01D 11/0288; B01D 21/262

(57) ABSTRACT

An extraction method of proanthocyanidins from Myrica rubra Sieb. Et Zucc leaves is provided. In the method, proanthocyanidins from Myrica rubra Sieb. Et Zucc leaf are obtained by preparing an aqueous Myrica rubra Sieb. Et Zucc leaf suspension and centrifuging. In the method, only water is used as an extraction reagent without introduction of an organic extraction solvent, which reduces costs and is free from environmental pollution; moreover, the method does not cause organic reagent residue in a product, such that the biological activity of the proanthocyanidins is not affected and product safety is improved without affecting product quality; meanwhile, extraction steps are simplified, extraction time is reduced and extraction efficiency is improved. The method provides stability, good reproducibility, simple equipment and easy control of conditions.

### CROSS-REFERENCE TO RELATED APPLICATION

This patent application claims the benefit and priority of Chinese Patent Application No. 202010494908.8, entitled “Extraction method of myrica rubra leaf proanthocyanidin” filed on Jun. 3, 2020, the disclosure of which is incorporated by reference herein in its entirety as part of the present application.

### TECHNICAL FIELD

The present disclosure relates to a rapid extraction technology of proanthocyanidins from Chinese bayberry (Myrica rubra Sieb. Et Zucc) leaves, in particular to a direct extraction method of proanthocyanidins from Myrica rubra Sieb. Et Zucc leaves using water as an extraction medium.

### BACKGROUND ART

Proanthocyanidins are polyphenols with C6-C3-C6 structure widely present in plants. They are also called condensed tannins They are the second largest type of natural phenols after lignin, which are widely consumed and can be taken up to 95 mg/day in European and American countries. Among them, main subunits of grape seed proanthocyanidins are catechin, epicatechin, epicatechin gallate, epigallocatechin gallate, and the like, with an average degree of polymerization greater than 10. Grape seed proanthocyanidins are internationally recognized as free radical scavengers and natural antioxidants in vivo, which are also popular nutritional supplements in the United States.

A most commonly used extraction method of proanthocyanidins is extraction with 70% acetone (Journal of Agricultural and Food Chemistry, 2011, 59, 1622-1629). Acetone is a toxic, flammable and explosive liquid, which can cause a lot of safety risks during extraction. In order to obtain proanthocyanidins with higher purity, extraction with hexane is also a common method, and hexane is also a flammable and toxic gas, which will cause many safety risks during extraction too (Oncology Letters, 2017, 13, 3210-3218).

### SUMMARY

In view of the shortcomings of the prior art, the present disclosure provides an extraction method of proanthocyanidins from Myrica rubra Sieb. Et Zucc leaves. The method can avoid involving acetone and hexane in extraction process of proanthocyanidins from Myrica rubra Sieb. Et Zucc leaves. The extraction method is simple with high extraction rate, and obtained product structure has not changed significantly.

An objective of the present disclosure is achieved by the following technical solution: an extraction method of proanthocyanidins from Myrica rubra Sieb. Et Zucc leaves, specifically including following steps:

step 1, adding a dried and ground M. rubra Sieb. Et Zucc leaf powder to 90-100° C. hot water and stirring evenly to obtain a M. rubra Sieb. Et Zucc leaf suspension;

step 2, putting the M. rubra Sieb. Et Zucc leaf suspension obtained in step 1 into a water bath at 90-100° C. and stirring for 30-90 min;

step 3, centrifuging the M. rubra Sieb. Et Zucc leaf suspension treated in the water bath in step 2 at 3,000-8,000 rpm for 10-30 min, and collecting a supernatant and a filter residue, respectively;

step 4, repeating steps 1 to 3 for the filter residue collected in step 3 for 2-4 times; and

step 5, mixing all supernatants and drying to obtain the proanthocyanidins from Myrica rubra Sieb. Et Zucc leaves.

Compared with the prior art, the present disclosure has following beneficial effects: The extraction method of proanthocyanidins from Myrica rubra Sieb. Et Zucc leaves provided by the present disclosure uses a method of similar miscibility to extract the water-soluble compound proanthocyanidins from M. rubra Sieb. Et Zucc leaves. In the method, only water is used as an extraction reagent without introduction of an organic extraction solvent, which reduces costs and is free from environmental pollution; moreover, the method does not cause organic reagent residue in a product, such that biological activity of the proanthocyanidins is not affected and product safety is improved without affecting the product quality; meanwhile, extraction steps are simplified, extraction time is reduced and extraction efficiency is improved. The method is characterized by stability, good reproducibility, simple equipment, easy control of condition, and suitability for any M. rubra Sieb. Et Zucc leaves.

### DETAILED DESCRIPTION OF THE EMBODIMENTS

### Example 1: Extraction of Proanthocyanidins from Myrica rubra cv. DongKui Leaves

Step 1, a dried and ground Myrica rubra cv. DongKui leaf powder was added to 90° C. hot water and stirred evenly to obtain a Myrica rubra cv. DongKui leaf suspension;

step 2, the Myrica rubra cv. DongKui leaf suspension obtained in step 1 was put into a water bath at 90° C. and stirred for 90 min;

step 3, the Myrica rubra cv. DongKui leaf suspension treated in the water bath in step 2 was centrifuged at 3,000 rpm for 10 min, and a supernatant and a filter residue were collected, respectively;

step 4, steps 1 to 3 were repeated for the filter residue collected in step 3 for twice; and

step 5, all supernatants were mixed and freeze-dried to obtain the proanthocyanidins from Myrica rubra cv. DongKui leaves.

A content of proanthocyanidin from Myrica rubra Sieb. Et Zucc leaves was detected by a vanillin method and reached more than 70%.

### Comparative Example 1: A Traditional Extraction Method of Proanthocyanidins from Myrica rubra cv. DongKui Leaves

Step 1, a dried and ground Myrica rubra cv. DongKui leaf powder was mixed with a mixture of water and acetone (in a volume ratio of 3:7), and stirred evenly;

step 2, the Myrica rubra cv. DongKui leaf suspension in step 1 was centrifuged at 3,000 rpm for 10 min, and a supernatant and a filter residue were collected, respectively;

step 3, steps 1 to 2 were repeated for the filter residue collected in step 3 for twice;

step 4, all supernatants were mixed and rotary evaporated under vacuum at 50° C. until solution no longer boiled and no pungent odor existed; the solution was removed and centrifuged at 8,000 rpm for 10 min, and the supernatant was collected; and

step 5, the supernatant in step 4 was freeze-dried to obtain a sample of proanthocyanidins from Myrica rubra cv. DongKui leaves.

A content of proanthocyanidins from Myrica rubra Sieb. Et Zucc leaves obtained by the foregoing method was detected by the vanillin method and reached more than 70%.

### Example 2: Extraction of Proanthocyanidins from Myrica Rubracv. Biqi Leaves

Step 1, a dried and ground Myrica rubracv. Biqi leaf powder was added to 100° C. hot water and stirred evenly to obtain a Myrica rubracv. Biqi leaf suspension;

step 2, the Myrica rubracv. Biqi leaf suspension obtained in step 1 was put into a water bath at 100° C. and stirred for 30 min;

step 3, the Myrica rubracv. Biqi leaf suspension treated in the water bath in step 2 was centrifuged at 8,000 rpm for 30 min, and a supernatant and a filter residue were collected, respectively;

step 4, steps 1 to 3 were repeated for the filter residue collected in step 3 for four times; and

step 5, all supernatants were mixed, rotary evaporated under vacuum, and dried to obtain the proanthocyanidins from Myrica rubracv. Biqi leaves.

A content of proanthocyanidins from Myrica rubra Sieb. Et Zucc leaves was detected by the vanillin method and reached more than 70%.

### Comparative Example 2: A Traditional Extraction Method of Proanthocyanidins from Myrica Rubracv. Biqi Leaves

Step 1, a dried and ground Myrica rubracv. Biqi leaf powder was mixed with a mixture of water and acetone (in a volume ratio of 3:7), and stirred evenly;

step 2, the Myrica rubracv. Biqi leaf suspension in step 1 was centrifuged at 8,000 rpm for 30 min, and a supernatant and a filter residue were collected, respectively;

step 3, steps 1 to 2 were repeated for the filter residue collected in step 3 for four times;

step 4, all supernatants were mixed and rotary evaporated under vacuum at 50° C. until the solution no longer boiled and no pungent odor existed; the solution was removed and centrifuged at 8,000 rpm for 10 min, and the supernatant was collected; and

step 5, the supernatant in step 4 was freeze-dried to obtain a sample of proanthocyanidins from Myrica rubracv. Biqi leaves.

A content of proanthocyanidins from Myrica rubra Sieb. Et Zucc leaves obtained by the foregoing method was detected by a vanillin method and reached more than 70%.

From two examples and two comparative examples above, it can be seen that the examples avoid pollution of organic reagents without using organic reagent acetone, but obtain proanthocyanidins from Myrica rubra Sieb. Et Zucc leaves of the same purity, and example experimental steps are simple and less time-consuming.

Finally, it should be noted that above examples are only a few specific examples of the present disclosure. Obviously, the present disclosure is not limited to above examples, and many variations are possible. All modifications that can be directly derived or imagined by a person of ordinary skill in the art from the disclosure herein should be deemed as falling within the claimed scope of the present disclosure.

1. An extraction method of proanthocyanidins from Myrica rubra Sieb. Et Zucc leaves, comprising:
adding a dried and ground Myrica rubra Sieb. Et Zucc leaf powder to 90-100° C. hot water and stirring evenly to obtain a Myrica rubra Sieb. Et Zucc leaf suspension;
putting the obtained Myrica rubra Sieb. Et Zucc leaf suspension into a water bath at 90-100° C. and stirring for 30-90 min;
centrifuging the Myrica rubra Sieb. Et Zucc leaf suspension heated in the water bath at 3,000-8,000 rpm for 10-30 min, and collecting a supernatant and a filter residue, respectively;
repeating said adding, stirring, putting, centrifuging, and collecting for the filter residue for 2-4 times; and
mixing and drying collected supernatants to obtain the proanthocyanidins from Myrica rubra Sieb. Et Zucc leaves.
